(19) United States
(12) Patent Application Publication
(10) Pub. No.: US 2012/0253432 A1
(43) Pub. Date: Oct. 4, 2012

(54) LIGHT TREATMENT SYSTEM

(75) Inventor: Damien Gerard Loveland, Richmond (CA)

(73) Assignee: KONINKLIJKE PHILIPS ELECTRONICS N.V., EINDHOVEN (NL)

(21) Appl. No.: 13/515,997

(22) PCT Filed: Dec. 13, 2010
(86) PCT No.: PCT/IB2010/055772

§ 371 Date: Jun. 14, 2012

(30) Foreign Application Priority Data
Dec. 16, 2009 (EP) 09179371.1

(51) Int. Cl. A61N 5/06

(52) U.S. Cl. 607/90

(57) ABSTRACT

The invention relates to a light treatment system (10, 12, 14). The light treatment system comprises an elastic sheet (20) connected to a deformable rim (30). The deformable rim comprises a perimeter having a substantially fixed length. The elastic sheet comprises light emitting means (40, 42, 44) configured for illuminating a treatment area (100), the elastic sheet being configured for being elastic in a direction substantially parallel to the elastic sheet for reducing the occurrence of folds in the elastic sheet when reshaping the deformable rim, and being configured for being conformable to the treatment area. An effect of the light treatment system according to the invention is that the elastic property of the elastic sheet enables the deformable rim to be deformed in substantially any direction while the elastic sheet remains substantially conformable to the treatment area. Consequently a substantial uniform illumination of the treatment area is ensured while allowing a well defined fit to substantially any body part.

### FIELD OF THE INVENTION

The invention relates to a light treatment system.

### BACKGROUND OF THE INVENTION

Light treatment systems are known per se. They are used, inter alia, in skin treatment systems for providing skin treatment for cosmetic and medical applications. Medical light treatment, for example, comprises light treatment using ultraviolet light radiation for treatment of, for example, psoriasis and, for example, red or blue light treatment, combined with a photo-synthesizer, for the treatment of, for example, skin-cancer. Cosmetic light treatments, for example, comprise skin rejuvenation using, for example, a mixture of infrared light and amber light. Such a skin rejuvenation process is, for example, described in the US patent U.S. Pat. No. 6,663,659.

Another system for skin treatment through illumination of light is known from WO 2006/081221 which discloses a phototherapy treatment device for applying area lighting to a wound. The disclosed phototherapy treatment device includes a light emitter that is adapted to be placed in close proximity to a wound for applying light/heat energy to the wound to aid in the healing process. The light emitter may comprise a light guide that receives light from a light source or a light source that is affixed to a substrate used to position the light source over the wound. The light guide may be flexible or the light sources may be arranged on a flexible circuit. This phototherapy device may be used at a treatment area which may be curved only in one direction, such as a part of a cylindrically shaped wound at an arm or a leg. However, when the treatment area is, for example, a part of a knee, shoulder or elbow, the known phototherapy treatment means may not properly work.

Specific bandages for applying phototherapy, for example, to a knee may be found in US 2005/0177093 in which slits have been applied to the flexible sheet such that the knee may be bent. However, as will be apparent from the illustrations in US 2005/0177093, not all of the knee-surface may be treated by the phototherapy device as the slits and holes, which allow the phototherapy device to obtain the three-dimensional shape of the knee, cannot be used to emit light.

A drawback of these known phototherapy treatment devices is that they are only well suited for a limited number of body parts.

### SUMMARY OF THE INVENTION

So it is an object of the invention to provide a light treatment device which may effectively be used for substantially all body parts of a human or animal body.

According to a first aspect of the invention the object is achieved with a light treatment system as claimed in claim 1.

The light treatment system according to the first aspect of the invention comprises an elastic sheet connected to a deformable rim, the deformable rim comprising a perimeter having a substantially fixed length, and the elastic sheet comprising light emitting means configured for illuminating a treatment area, the elastic sheet being configured for being elastic in a direction substantially parallel to the elastic sheet for reducing the occurrence of folds in the elastic sheet when reshaping the deformable rim and being configured for being conformable to the treatment area.

An effect of the light treatment system according to the invention is that, when applied to a treatment area, the elastic property of the elastic sheet enables the deformable rim to be deformed in substantially any direction, thereby conforming the rim to the body shape surrounding the treatment area, while the elastic sheet remains substantially conformed to the surface of the treatment area. Consequently a substantial uniform illumination of the treatment area is ensured while allowing a well defined fit to substantially any body part. The light emitting means illuminating the treatment area should preferably be arranged substantially parallel to the treatment area. When the treatment area is part of a human or animal body, the treatment area may have any three-dimensional shape. Due to the elastic property of the elastic sheet in the light treatment system according to the invention, the light treatment system may be applied to the light treatment area having substantially any shape while maintaining the light emitting means positioned substantially conform to the treatment area. The elastic property of the elastic sheet may also reduce the occurrence of folds in the sheet comprising the light emitting means, which may occur when trying to conform to the curved three-dimensional shape of the treatment area, and it may even prevent the occurrence of these folds. Such folds may alter the light distribution across the treatment area considerably which may hurt the patient rather than contribute to the curing of the patient with the light treatment. In the light treatment system according to the invention the combination of a deformable rim having a perimeter with a substantially fixed length and an elastic sheet comprising the light emitting means enables the light treatment system to be applied to a treatment area having substantially any shape.

Light emitted by the light emitting means for light treatment may comprise light visible to the naked human eye and/or may comprise light invisible to the naked human eye, such as infrared light and/or ultraviolet light. The light emitting means may also be configured for emitting light having a relatively broad emission spectrum which may comprise a substantially continuous spectrum over a predefined range or which may comprise a discontinuous spectrum over the predefined range. Such a discontinuous spectrum may comprise a plurality of emission peaks from light emitters inside the light emitting means. Such light emitters may, for example, emit light around a central wavelength having a relatively narrow spectral peak. Light emitters emitting a relatively narrow spectral peak, for example, comprise semiconductor light emitters such as light emitting diodes, laser diode and/or organic light emitting diodes. Light emitters emitting a relatively broad and substantially continuous spectrum may comprise all types of lamps emitting a relatively broad spectrum. The emission spectrum of a light emitter may, for example, be enhanced via the use of luminescent materials.

In an embodiment of the light treatment system, the elastic sheet is configured to comprise a tensile force, acting locally in a direction substantially parallel to the elastic sheet and existing substantially within a range of deformability of the deformable rim useful for treatment. Due to the substantial continuous presence of the tensile force over a range of deformable rim shapes, the elastic sheet may always be substantially smooth and non-rippled, even while reshaping the deformable rim into substantially any shape. The tensile force is present within a reshaping range of the deformable rim useful for treatment. The useful range of rim shapes for treatment comprises a range in which body parts, or parts thereof, are covered by the elastic sheet and substantially uniform illumination of that body part is provided. Excessive reshaping such that, for example, parts of the deformable rim touch each other are clearly outside the reshaping range useful for treatment and in such excessive reshaping no tensile force may be present. A minimum distance, measured along the surface of the elastic sheet, between opposing parts of the deformable rim which is equal or larger than two times the sum of the width dimension of the light emitting means arranged between these parts may be required to maintain some residual tensile force present in the elastic sheet to substantially prevent the elastic sheet from rippling. The width dimension of the light emitting means is a dimension of the light emitting means locally in a direction substantially parallel to the elastic sheet. In another embodiment, features of the deformable rim may limit the rim deformation with a certain minimum radius of curvature, or different minima depending on whether a portion of the rim is curved inwards or outwards. In such an embodiment, the presence of folds in the elastic sheet due to the reshaping of the deformable rim is fully prevented thus ensuring a good fit to the treatment area. The tensile force may be present in the elastic sheet due to the fact that it is continuously stretched by the deformable rim, which may be configured for having a perimeter comprising a substantially fixed length and a perimeter shape within a range of deformable shapes. That is, the deformable rim may have any shape as long as some residual tensile force still is present at the minimum dimension of the elastic sheet. The minimum dimension of the elastic sheet is defined as the minimum distance between two sides of the deformable rim measured along the elastic sheet. This residual tensile force may be present to prevent folding of the elastic sheet.

In an embodiment of the light treatment system, the deformable rim constitutes an outer rim of the elastic sheet. This embodiment has as an advantage that the combination of the deformable rim with the elastic sheet may constitute a closed environment in which the light treatment occurs. The borders of such closed environment may comprise the treatment area the deformable rim and the elastic sheet. An advantage of such a closed environment may be to prevent, for example, light from leaking away from the treatment area. Some light treatment is done using, for example, ultraviolet light which may be harmful to the human or animal eye. By using the light treatment system in which the environment to be illuminated is sealed, the harmful radiation may be confined to the treatment area only. The environment may, alternatively, also be closed to control the presence of fluids on or around the treatment area while applying the light treatment. Photo-synthesizers may be applied which may react with the applied light of the light treatment system to provide the light treatment. In such closed environment the photo-synthesizer may be applied as a fluid inside the closed environment or by having the fluids flow through the closed environment.

In an embodiment of the light treatment system, the elastic sheet is configured for preventing transmission of at least part of the light emitted from the light emitting means. As indicated before, some light treatment is performed using ultraviolet light which may be damaging to the human or animal eye. The elastic sheet may, for example, be configured to prevent transmission of the harmful ultraviolet light while allowing for example infrared radiation to pass through the elastic sheet. While applying light treatment, the treatment area may become relatively hot. By allowing part of the infrared radiation to pass through the elastic sheet, some of the heat may irradiate away from the treatment area while confining the ultraviolet light to the treatment area only.

In an embodiment of the light treatment system, the elastic sheet comprises a reflective layer for reflecting at least part of the light emitted from the light emitting means or scattered around toward the treatment area. This reflection layer may enhance the efficiency of the light treatment system as any light not absorbed by the treatment area but reflected away from the treatment area may be reflected back towards the treatment area via the reflective layer. This increased efficiency enables to reduce the cost of the light treatment system because light emitting means may be used having lower intensity to achieve effective light treatment.

In an embodiment of the light treatment system, the light emitting means comprises a light source or an array of light sources connected to the elastic sheet. This connection may comprise some kind of attachment means which may include glue for connecting the light source or the individual light sources in the array of light sources to the elastic sheet. Alternatively, the light source or the individual light sources of the array of light sources may be inserted in holes in the elastic sheet. Such holes may be relatively small such that the elastic sheet will be locally stretched to fit around the outer dimensions of the light source. The edges of the light source may comprise a groove in which the edge of the holes may fit to connect the light source to the elastic sheet. The groove may, for example, be part of a heat sink of the light source.

In an embodiment of the light treatment system, the light emitting means comprises an array of light sources connected to the elastic sheet and at least one of the light sources in the array of light sources is for emitting light comprising a different wavelength compared to the remainder of the light sources in the array of light sources. Such a light treatment system may be used in a light treatment in which light of different wavelengths may be required for the treatment. For example, by having some of the light sources in the array of light sources to emit infrared light while the remainder of the light sources emit ultraviolet light, the temperature of the treatment area at which the light treatment takes effect may be regulated. The penetration depth of ultraviolet light strongly depends on the width of the pores on the human or animal skin. By using infrared light sources, the temperature of the treatment area at the human or animal skin may be elevated such that the pores in the human or animal skin open which improves the penetration depth of the ultraviolet light, thus improving the efficiency of the light treatment. Of course, any other combinations of light sources may be used.

In an embodiment of the light treatment system, the light emitting means may comprise light conversion means for converting light impinging on the conversion means into light of a different wavelength. Light conversion means may be used to shift and/or broaden the emission spectrum of the light emitted by a light source. When, for example, light emitting diodes are used as light emitting means, the width of the spectrum emitted from a light emitting diode is relatively narrow. Using light conversion means, the spectrum may be broadened, which may, for example, be required for obtaining an efficient light treatment. The conversion means may be applied directly on the light sources or may be arranged between the light sources and the treatment area, for example, applied as a layer on the elastic sheet or as a remote phosphor on a separate cover sheet (see further in the description for an explanation of the function of a cover sheet). Alternatively, the conversion means may be present as indicator that a specific type of light source is emitting light. For example, the use of ultraviolet light may be harmful to the human or animal eye. The elastic sheet may block the ultraviolet light; however, as the ultraviolet light is invisible to the human or animal eye, it may not be directly apparent that ultraviolet light emission has started. By applying a light conversion means in the neighborhood of the light source, part of the ultraviolet light may be converted by the conversion means into visible light functioning as a warning system to warn any user that ultraviolet light emission has started.

In an embodiment of the light treatment system, the light emitting means comprises light extraction means arranged in the elastic sheet constituting a light guiding medium. The elastic sheet may be the light guiding medium which is elastic in a direction substantially parallel to the elastic sheet. The extraction of light from the light guiding medium may be achieved using light extraction means well known in the art, such as scattering means, diffusing means or reflecting means. Preferably the light guide confines the light using total internal reflection to ensure a substantially loss-less confinement of light. This embodiment may only be feasible for a very specific wavelength range of the light emitted by the light emitting means. This is caused by the fact that most elastic material is produced from polymer material which often is damaged by light, especially by ultraviolet light.

In an embodiment of the light treatment system, the elastic sheet comprises spacing means configured for maintaining the flexible sheet at a predefined distance from the treatment area. The spacing means may comprise of local projections from the elastic sheet having a specific height defining the predefined distance. The height of the projection is a dimension of the projection measured in a direction substantially perpendicular to the local surface of the elastic sheet. Alternatively, local walls or dimples may be present having the specific height for defining the predefined distance. Walls may, for example, be present substantially surrounding individual light sources limiting distribution of the light emitted from the individual light source.

In an embodiment of the light treatment system, the deformable rim comprises resilient material for contacting a surrounding of the treatment area so as to prevent light from leaking away from the treatment area and/or for generating a substantially sealed environment around the treatment area. The resilient material may be used to ensure that there is a close fit between the deformable rim and the surrounding of the treatment area. This close fit may be used only to prevent light from leaking away. Alternatively, the resilient material may also be used to seal the treatment area from the surrounding and confine a fluid to the environment being enclosed by the deformable rim and the resilient material, the elastic sheet and the treatment area. In this way, the treatment area may be flooded with a fluid which may be used during light treatment. Such resilient material may also be used to enhance the comfort of the human or animal on which the light treatment is performed.

In an embodiment of the light treatment system, the deformable rim comprises a mechanical-snake-like structure constituted by connecting joints being enclosed in a flexible casing being adhered to the flexible sheet. Such mechanical-snake-like structures, as for example used in flexible stems of desk lamps, allow the perimeter of the deformable rim to remain substantially constant while allowing the deformable rim to be shaped into substantially any three-dimensional shape. The joints help to define the range within which the deformable rim may be deformed. In an embodiment of the light treatment system, the joints comprise a predefined level of friction for maintaining a shape of the deformable rim after reshaping. This level of friction preferably is larger than the elastic force applied to the deformable rim by the elastic sheet. In a preferred embodiment of the elastic sheet, the elastic sheet comprises a tension force substantially within the range of deformability of the deformable rim useful for treatment. When the level of friction is sufficient to withstand this tension force, the light treatment system may be shaped in substantially any shape possible by the deformable rim, maintaining this shape while avoiding any folds to occur in the elastic sheet comprising the light emitting means.

In an embodiment of the light treatment system, the light treatment system comprises a translucent cover sheet arranged between the elastic sheet and the treatment area. Such translucent cover sheet may be used to shield the light emitting means from touching the treatment area. Often light emitting means such as light emitting diodes may have elevated temperature. Such locally elevated temperature may not be pleasant for the human or animal undergoing the light treatment. Using such translucent cover sheet between the elastic sheet and the treatment area may prevent direct contact between the light emitting means and the treatment area. The cover sheet may also comprise a remote phosphor to convert light from the light emitters to other wavelength ranges suitable for the light therapy, as mentioned above.

In an embodiment of the light treatment system, the translucent cover sheet is removably attached to the light treatment system. The translucent cover sheet may be applied to the deformable rim via an additional rim or groove around the deformable rim. Due to the removably attached translucent cover sheet, the light treatment system may be reused for light treatment on different humans. Some light treatments may be required for illnesses which may be transferable from one patient to another. When the light treatment system may be used for more than one patient, the light treatment system may contaminate another patient. Using the translucent cover sheet may ensure that substantially no contamination is possible from one patient to the other. By replacing the translucent cover sheet before applying the light treatment via the light treatment system to a next patient, any possible contamination from one patient to the other is avoided. This strongly enhances the cost efficiency of the light treatment system as it may be used for multiple patients by simply replacing the translucent cover sheet. In a preferred embodiment, also the translucent cover sheet is configured for comprising a tensile force locally in a direction substantially parallel to the translucent cover sheet and existing substantially within the range of deformability of the deformable rim useful for treatment to reduce the occurrence of folds due to the deforming of the translucent sheet via deforming of the deformable rim, or even prevent the occurrence of folds due to the deforming of the deformable rim.

In an embodiment of the light treatment system, the light treatment system comprises fastening means for attaching the system to a part of the human or animal body comprising the treatment area. These fastening means may comprise straps which may, for example, be partially elastic and which may be fastened using Velcro or similar fastening means.

In an embodiment of the light treatment system, the light treatment system comprises a control circuit being configured for controlling the light emitting means for emitting light towards the treatment area, the control circuit being configured for controlling an intensity, and/or a duration, and/or a wavelength, and/or a frequency in a pulse-mode operation, and/or a dead-time in a pulse-mode operation. The sequence and/or intensity via which light is applied during the light treatment to the treatment area may be different for the different light treatments. Having the control circuit, the light treatment system may be configured for applying the light to the treatment area in the applied sequence and/or intensity.

In an embodiment of the light treatment system, the light treatment system comprises vacuum means for applying a reduced pressure inside a sealed environment comprising the elastic sheet, the deformable rim and the treatment area. Such a vacuum means may be used to ensure a tight fit between the light treatment system and the human or animal skin to which the light treatment is applied. Especially in combination with spacing means for maintaining the flexible sheet at the predefined distance from the treatment area, the use of the vacuum means allows a well defined fit to substantially any body part, including concave body parts.

An embodiment according to the invention may comprise multiple deformable rims attached or arranged so as to create a grid or network of deformable rims. Each of the deformable rims may have properties similar to the deformable rim described above, e.g. a substantially fixed perimeter length, and may share part of their perimeter with other deformable rims. An example of such a deformable rim network may be a (deformable) honeycomb structure. A single elastic sheet, comprising one or more light emitters, may be connected to the deformable rim network. Alternatively multiple elastic sheets, each comprising one or more light emitters, may be connected to the deformable rim network. Continuing with the honeycomb example, one elastic sheet may be used per (deformable) honeycomb cell. The deformable rim network allows for even more flexibility towards body part shapes that can be covered smoothly by the light treatment system according to the invention. For example, the deformable rim network may be deformed in a fully concave shape for covering concave body parts, without requiring vacuum means, as explained in previous paragraph, to realize the close fit with the concave body part. It is to be noted that due to the tensile forces in the elastic sheet, embodiments of light treatment systems having a single deformable rim are already capable of smoothly covering a combined convex/concave surface such as for example a horse saddle (e.g. a knee or elbow area) or a fully concave surface with the aid of vacuum means.

### BRIEF DESCRIPTION OF THE DRAWINGS

These and other aspects of the invention are apparent from and will be elucidated with reference to the embodiments described hereinafter.

In the drawings:

FIG. 1 is a schematic cross-sectional view of an embodiment of the light treatment system according to the invention,

FIGS. 2A and 2B shows possible deformations of the light treatment system according to the invention,

FIGS. 3A to 3H show different constructional details of an embodiment of a light treatment system according to the invention, and

FIG. 4 shows an embodiment of a light treatment system according to the invention applied to an elbow of a person.

The figures are purely diagrammatic and not drawn to scale. Particularly for clarity, some dimensions are exaggerated strongly. Similar components in the figures are denoted by the same reference numerals as much as possible.

### DETAILED DESCRIPTION OF EMBODIMENTS

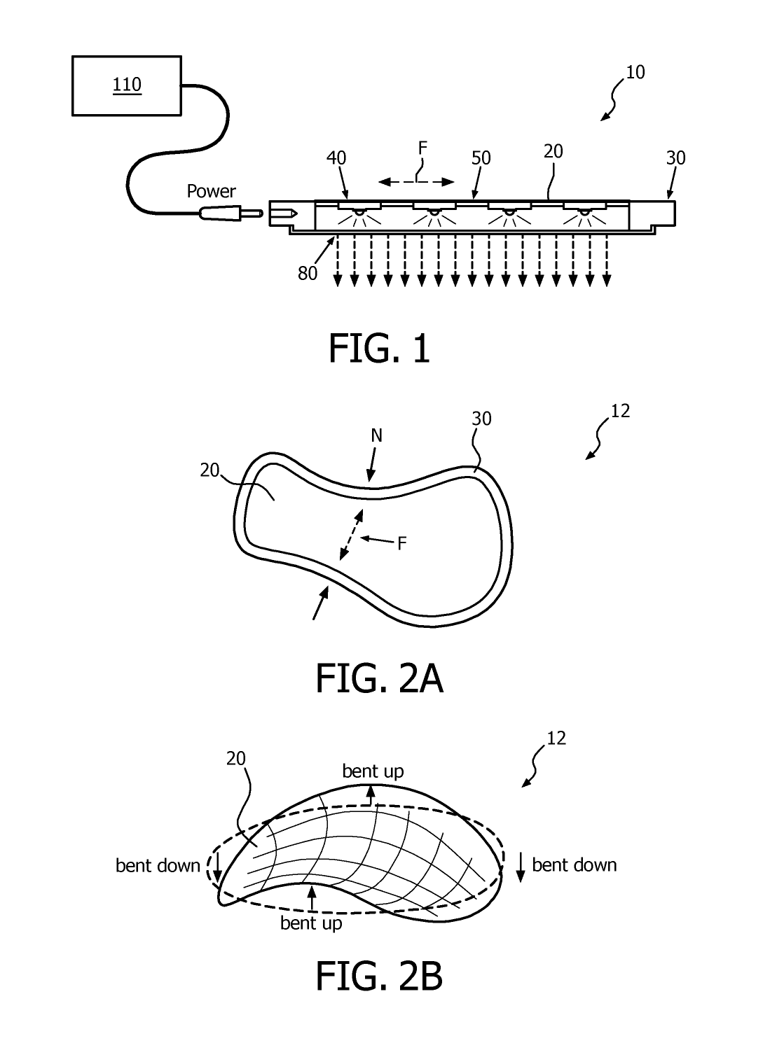

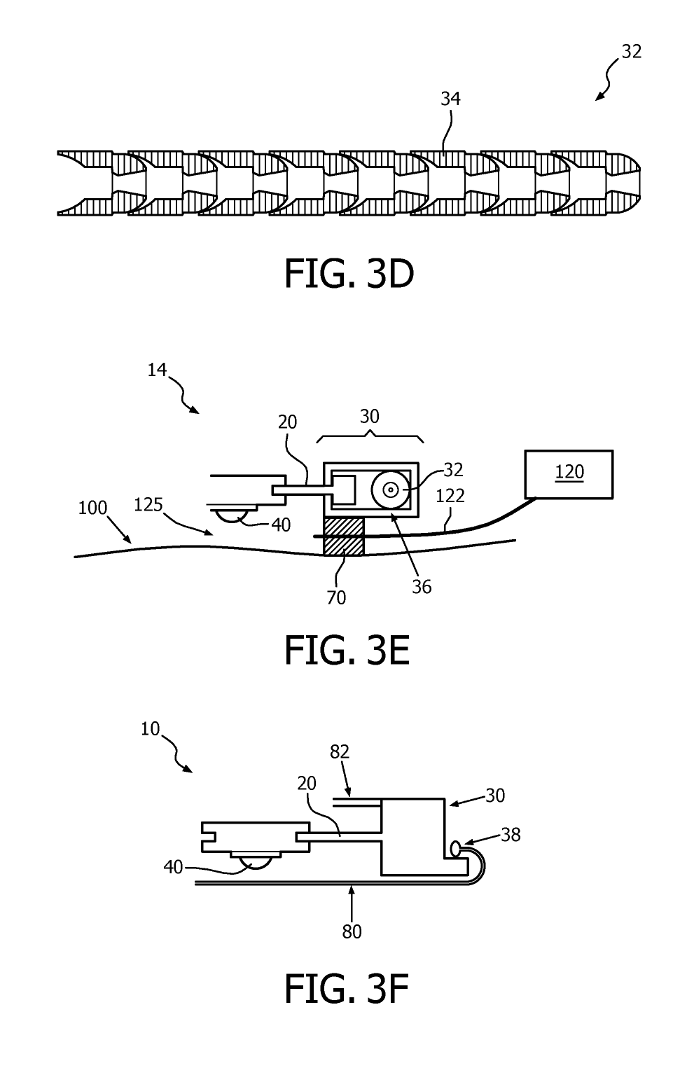

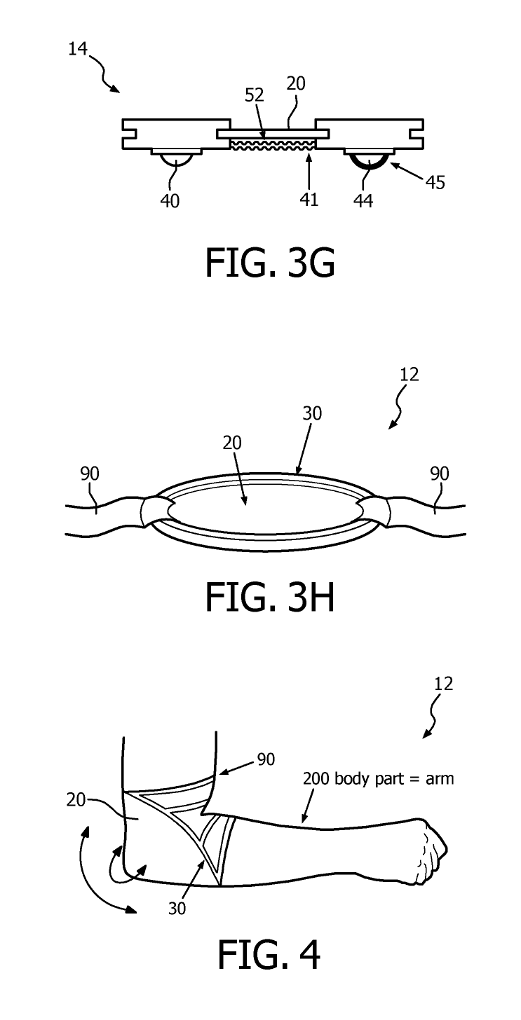

FIG. 1 is a schematic cross-sectional view of an embodiment of the light treatment system 10 according to the invention. The light treatment system 10 comprises an elastic sheet 20 connected to a deformable rim 30. The deformable rim 30 comprises a perimeter having a substantially fixed length. The elastic sheet 20 comprises light emitting means 40 configured for illuminating a treatment area 100 (see FIG. 3A). The elastic sheet 20 is configured for being elastic in a direction substantially locally parallel to the elastic sheet 20. The use of the elastic sheet 20 reduces the occurrence of folds in the surface on which the light emitting means 40 are applied when reshaping the elastic sheet 20 and/or the deformable rim 30 or even prevents the occurrence of folds in the sheet 20. As a result, a substantial uniform illumination of the treatment area 100 is ensured while allowing the light treatment system 10 to fit well to substantially any body part 200 (see FIG. 4). The light emitting means 40 which illuminate the treatment area 100 are preferably arranged substantially parallel to the treatment area 100. However, when the treatment area 100 is part of the human or animal body 200 (for example, the elbow as shown in FIG. 4) the treatment area 100 may have any three-dimensional shape. Due to the elastic property of the elastic sheet 20 the light treatment system 10 may be applied to the light treatment area 100 having substantially any shape while maintaining the light emitting means 40 substantially parallel to the treatment area 100.

The light emitting means 40 may comprise a light source 40 or an array of light sources 40 connected to the elastic sheet 20. This connection may comprise some kind of attachment means (not shown) such as glue for connecting the light source 40 or the individual light sources 40 in the array to the elastic sheet 20. Alternatively, the light source 40 or the individual light sources 40 in the array may be connected to holes 43 (see FIG. 3C) in the elastic sheet 20. Such holes 43 may be relatively small such that the elastic sheet 20 is locally stretched to fit around the outer dimensions of the light source 40. In an embodiment of the light treatment system 10, at least one of the light sources 40 in the array emits light comprising a different wavelength compared to the remainder of the light sources 40 in the array. Such a light treatment system 10 may be used in a light treatment in which light of different wavelengths may be required for the treatment. For example, by having some of the light sources 40 in the array of light sources to emit infrared light while the remainder of the light sources 40 emit ultraviolet light, the temperature of the treatment area 100 at which the light treatment takes effect may be regulated. The penetration depth of ultraviolet light strongly depends on the width of the pores on the human or animal skin. By using infrared light sources, the temperature of the treatment area 100 at the human or animal skin may be elevated such that the pores in the human or animal skin open which improve the penetration depth of the ultraviolet light, improving the efficiency of the light treatment. Of course, any other combinations of light sources 40 may be used.

The light treatment system may also comprise a control circuit 110 for controlling the light emitting means 40 for emitting light towards the treatment area 100. The control circuit may be configured for controlling an intensity, and/or a duration, and/or a wavelength, and/or a frequency in a pulse-mode operation, and/or a dead-time in a pulse-mode operation. The sequence and/or intensity via which light is applied during the light treatment to the treatment area 100 may be different for the different light treatments. The control circuit 110 may ensure that the light treatment is applied in a specific required sequence and/or intensity.

The elastic sheet may, for example, consist in whole or in part of, for example, rubber, nitrile rubber, neoprene, latex, polyisoprene. The elasticity of this material prevents the elastic sheet 20 which comprises the light emitting means 40 from folding due to the three-dimensional shape of the treatment area 100. Such folds would alter the light distribution across the treatment area 100 considerably. So the combination of a deformable rim 30 having a perimeter with a substantially fixed length with the elastic sheet 20 comprising the light emitting means 40 enables the light treatment system to be applied to a treatment area 100 having substantially any shape.

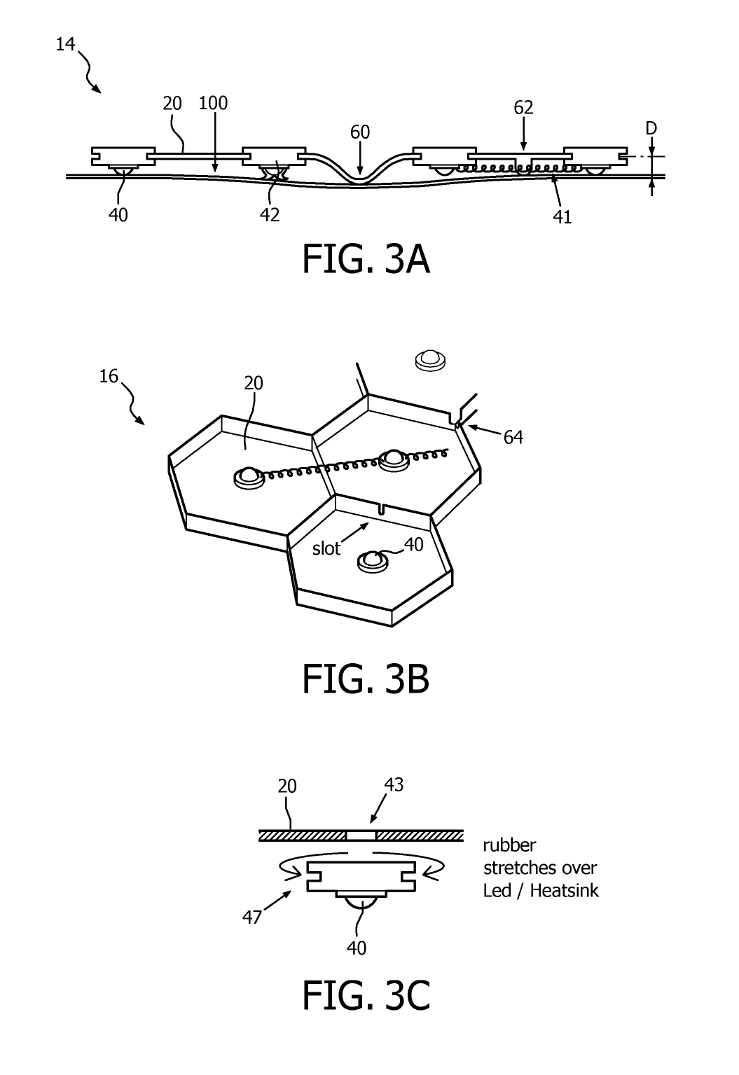

The elastic sheet 20 may, for example, be configured for comprising a tensile force F (indicated with a dashed double arrow in FIG. 1) in a direction locally substantially parallel to the elastic sheet 20 substantially within a whole range of deformability of the deformable rim 30. In a preferred embodiment the tensile force F is present in the elastic sheet 20 within a reshaping range of the deformable rim 30 useful for treatment. Due to the substantial continuous presence of the tensile force F, the elastic sheet 20 may always be substantially unrippled while reshaping the deformable rim 30. The tensile force F may be present in the elastic sheet 20 due to the fact that the deformable rim 30 is configured for having a perimeter comprising a substantially fixed length. The deformable rim 30 may have any shape (see for example, FIG. 2A) useful for treatment. Preferably some residual tensile force F may still be present at the minimum dimension of the shape (as is shown in FIG. 2A) to reduce the occurrence of folds or even prevent folds to occur.

The elastic sheet 20 may comprise means for preventing transmission of at least part of the light emitted from the light emitting means 40 outside the light treatment system, for example, a reflection layer 50 to prevent transmission of the harmful ultraviolet light. At the same time, this reflection layer 50 may be configured for allowing infrared radiation to pass through the elastic sheet 20 to limit the temperature increase at the treatment area 100.

The light treatment system 10 according to the invention may also comprise a translucent cover sheet 80 arranged between the elastic sheet 20 and the treatment area 100. Such translucent cover sheet 80 may be used to shield the light emitting means 40 from touching the treatment area 100 preventing contamination of the light treatment system 10 from the treatment area 100.

FIGS. 2A and 2B shows possible deformations of the light treatment system 12 according to the invention. Due to the presence of the tensile force F in a direction substantially parallel to the elastic sheet 20 the elastic sheet 20 does not start folding even not at the narrow deformed area N shown in FIG. 2A. The deformable rim 30 may have a predefined stiffness such that it maintains the reshaped form, even while the tensile force F is present in the elastic sheet 20. As shown in FIG. 2B also complex three-dimensional shapes may be generated by the deformable rim 30 resulting in a corresponding shape of the elastic sheet 20 which maintains stretched between the deformable rim 30—thus reducing the occurrence of folds or even preventing folds.

FIGS. 3A to 3H show different constructional details of embodiments of light treatment systems 10, 14, 16 according to the invention. FIG. 3A is a cross-sectional view showing spacing means 60, 62 for ensuring that the flexible sheet 20 is maintained at a predefined distance D from the treatments area 100. The spacing means 60, 62 may comprise local projections 62 or deformations 60 from the elastic sheet 20 having a specific height defining the predefined distance D. The height or predefined distance D of the spacing means 60, 62 is a dimension of the spacing means 60, 62 measured in a direction substantially perpendicular to the local surface of the elastic sheet 20. FIG. 3B shows alternative spacing means 64 being local walls 64 having the specific height for defining the predefined distance D. The walls 64 may, for example, surround individual light sources 40 to limit the distribution of the light emitted from the individual light source 40. In FIG. 3A it is further indicated that the individual light sources 40 in the array of light sources 40 may be connected via a wire 41 which preferably is flexible to allow the elastic sheet 20 to stretch while the light sources 40 remain connected via the wire 41.

FIG. 3C shows a possible manner in which the light source 40 may be attached to the elastic sheet 20. The light source 40 may comprise grooves 47 at the rim of the light source in which the elastic sheet 20 may be fitted. Holes 43 in the elastic sheet 20 may be used to mount the light source 40 into the elastic sheet 20 by locally stretching the elastic sheet 20 around the light source 40 to fit into the groove 47. Of course, other means for attaching the light source 40 to the elastic sheet 20 may be applied without departing from the scope of the invention, for example, using glue to attach the light source 40 to the elastic sheet 20.

FIG. 3D shows an example of a mechanical snake-like structure 32 for use in the deformable rim 30. The mechanical-snake-like structure 32 may, for example, be constituted by connecting joints 34 which may be held together using a flexible casing 36 (see FIG. 3E) to which the flexible sheet 20 is adhered. Similar mechanical-snake-like structures 32 may already be used, for example, in flexible stems of desk lamps (not shown). The construction of the connecting joints 34 allow the perimeter of the deformable rim 30 to remain substantially constant while allowing the deformable rim 30 to be shaped into substantially any three-dimensional shape. The joints 34 may, preferably, comprise a predefined level of friction for maintaining the shape of the deformable rim 30 after reshaping. This level of friction preferably is larger than the elastic force F applied to the deformable rim 30 by the elastic sheet 20. There may be an elastic cord in tension passing through the centre of the elements forming the mechanical-snake-like structure 32, which may also help control the amount of friction between the joints.

FIG. 3E shows part of a cross-sectional view of the light treatment system 14 comprising the mechanical-snake-like structure 32 embedded in the flexible casing 36 to which the elastic sheet 20 is connected. In the cross-sectional view of FIG. 3E the deformable rim 30 also comprises resilient material 70 for contacting a surrounding of the treatment area 100 for, for example, preventing light to leak away from the treatment area 100, and/or for generating a substantially sealed environment 125 around the treatment area 100. The borders of such closed environment 125 may comprise the treatment area 100 the deformable rim 30 and the elastic sheet 20. The closed environment 125 in FIG. 3E further comprises the resilient material 70 for contacting the surrounding of the treatments area 100 and to ensure that there is a good seal between the light treatment system 14 and the human or animal skin. The closed environment 125 may be used to control the presence of fluids (not shown) on or around the treatment area 100 while applying the light treatment. Photo-synthesizers (not shown) may be applied which may react with the applied light of the light treatment system 14 to provide the light treatment. In such closed environment 125 the photo-synthesizer may be applied as a fluid inside the closed environment 125 or by having the fluids flow through the closed environment 125.

The embodiment of the light treatment system 14 shown in FIG. 3E further includes vacuum means 120 for applying a reduced pressure inside a sealed environment 125. Such a vacuum means 120 may be used to ensure a tight fit between the light treatment system 14 and the human or animal skin to which the light treatment is applied. Especially in combination with spacing means 60, 62, 64 (see FIGS. 3A and 3B) for maintaining the flexible sheet 20 at the predefined distance D (see FIG. 3A) from the treatment area 100, the use of the vacuum means 120 allows a well defined fit to substantially any body part 200.

FIG. 3F shows part of a cross-sectional view of a further embodiment of the light treatment system 10 comprising the translucent cover sheet 80 which is removably attached to the light treatment system 10. The translucent cover sheet 80 may be applied to the deformable rim 30 via an additional rim 38 around the deformable rim 30. Due to the removably attached translucent cover sheet 80, contamination of the light treatment system 10 may be prevented enabling the light treatment system 10 to be reused for light treatment on different humans. By replacing the translucent cover sheet 80 before applying the light treatment via the light treatment system 10 to a next patient, any possible contamination from one patient to another is avoided. In a preferred embodiment, also the translucent cover sheet 80 is configured for comprising a tensile force in a direction substantially parallel to the translucent cover sheet 80 substantially within the range of deformability of the deformable rim 30 useful for treatment, similar to the elastic sheet 20. This prevents folds occurring in the translucent cover sheet 80 when deforming the deformable rim 30. The light treatment system 10 may further comprise a back-cover 82 which may be used to, for example, hide connection wires between light sources 40 from view and/or may be used to block any remaining ultraviolet light from leaking away from the light treatment system 10. The back-cover 82 may also be used for aesthetic appearance of the light treatment system 10 according to the invention.

FIG. 3G shows part of a cross-sectional view of a further embodiment of the light treatment system 14 comprising a plurality of light sources 40, 44 which are arranged in the elastic sheet 20 and connected via a wire 41. The cross-sectional view of FIG. 3G further shows reflection means 52 for reflecting light which may be scattered from the light treatment area 100 back towards the light treatment area 100 for enhancing the efficiency of the light treatment system 14. The reflection means 52 may only be reflective to part of the spectrum of the light emitted by the light sources 40, 44 in the light treatment system 14, and it may transmit other wavelengths. For example, when ultraviolet light is used, the reflection means 52 may reflect the ultraviolet light but may transmit other light such as infrared light used to heat up the treatment area 100.

The light emitting means 44 shown in FIG. 3G also comprises light conversion means 45 for converting light impinging on the conversion means 45 into light of a different wavelength. Light conversion means 45 may be used to shift and/or broaden the emission spectrum of the light emitted by a light source 40, 44. When, for example, light emitting diodes 40, 44 are used as light emitting means 40, 44, the width of the spectrum emitted from a light emitting diode 40, 44 is relatively narrow. Using light conversion means 45, the spectrum may be broadened, which may, for example, be required for obtaining an efficient light treatment. The conversion means 45 may be applied directly on the light sources 44 (as shown in FIG. 3G), or may be applied on some other surface in the light treatment system 14 (not shown). Alternatively, the conversion means 45 may be present as indicator that a specific type of light source 40, 44 is emitting light. For example, the use of ultraviolet light may be harmful to the human or animal eye. The elastic sheet may block the ultraviolet light; however, as the ultraviolet light is not visible to the human or animal eye, it may not be directly apparent that ultraviolet light emission has started. By applying a light conversion means 45 in the neighborhood of the light source 40, 44, part of the ultraviolet light may be converted by the conversion means 45 into visible light functioning as a warning system to warn any user that ultraviolet light emission has started.

FIG. 3H is a view of an embodiment of the light treatment system 12 comprising fastening means 90 for attaching the light treatment system 12 to a part of the human or animal body comprising the treatment area 100. These fastening means 90 may comprise straps 90 which may, for example, be partially elastic and which may be fastened using Velcro or similar fastening means.

FIG. 4 shows an embodiment of a light treatment system 12 according to the invention applied to a body part 200 of a person, being the elbow of a person. Due to the elastic property of the elastic sheet 20, the light treatment system 12 may be applied around the elbow of the person without causing parts of the elastic sheet 20 comprising the light sources 40 to fold. If the elastic sheet 20 which comprises the light source 40 folds, the distribution of light across the light treatment area 100 is not well defined anymore, causing the light treatment to be inefficient or even causing locally too high light intensities which may lead to local burns of the light treatment area 100. Consequently, the use of the elastic sheet 20 causes a good fit around substantially any body part 200 generating a broad application area of the light treatment system 12 according to the invention.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements. In the device claim enumerating several means, several of these means may be embodied by one and the same item of hardware. The mere fact that certain measures are recited in mutually different dependent claims does not indicate that a combination of these measures cannot be used to advantage.

1. A light treatment system (10, 12, 14) comprising an elastic sheet (20) connected to a deformable rim (30),
the deformable rim (30) having a perimeter comprising a substantially fixed length, and
the elastic sheet (20) comprising light emitting means (40, 42, 44) configured for illuminating a treatment area (100), the elastic sheet (20) being further configured for being elastic in a direction substantially parallel to the elastic sheet (20), for reducing the occurrence of folds in the elastic sheet (20) when reshaping the deformable rim (30) and being conformable to the treatment area (100).

2. The light treatment system (10, 12, 14) according to claim 1, wherein the elastic sheet (20) is configured for comprising a tensile force locally in a direction substantially parallel to the elastic sheet (20) and existing substantially within a range of deformability of the deformable rim (30) useful for treatment.

3. The light treatment system (10, 12, 14) according to claim 1, wherein the deformable rim (30) constitutes an outer rim of the elastic sheet (20).

4. The light treatment system (10, 12, 14) according to claim 3, wherein the elastic sheet (20) is configured for preventing transmission of at least part of the light emitted from the light emitting means (40, 42, 44).

5. The light treatment system (10, 12, 14) according to claim 4, wherein the elastic sheet (20) comprises a reflection layer (50, 52) for reflecting the at least part of the light emitted from the light emitting means (40, 42, 44) or scattered around towards the treatment area (100).

6. The light treatment system (10, 12, 14) according to claim 1, wherein the light emitting means (40, 42, 44) comprises:
a light source (40, 42, 44) connected to the elastic sheet (20), and/or
an array of light sources (40, 42, 44) connected to the elastic sheet (20), and/or
an array of light sources (40, 42, 44) connected to the elastic sheet (20), at least one of the light sources (40, 42, 44) in the array of light sources (40, 42, 44) emitting light comprising a different wavelength compared to the remainder of the light sources in the array of light sources (40, 42, 44), and/or
light conversion means (45) for converting light of a wavelength or wavelength range which impinges on the conversion means (45) into light of a different wavelength or wavelength range, and/or
light extraction means arranged in the elastic sheet (20) constituting a light guiding medium.

7. The light treatment system (10, 12, 14) as claimed in claim 6, wherein the elastic sheet (20) comprises spacing means (60, 62, 64) configured for maintaining the elastic sheet (20) at a predefined distance from the treatment area (100).

8. The light treatment system (10, 12, 14) according to claim 1, wherein the deformable rim (30) comprises resilient material (70) for contacting a surrounding of the treatment area (100) for preventing light from leaking away from the treatment area (100) and/or for generating a substantially sealed environment around the treatment area (100).

9. The light treatment system (10, 12, 14) according to claim 1, wherein the deformable rim (30) comprises a mechanical-snake-like structure (32) constituted by connecting joints (34) being enclosed in a flexible casing (36) being adhered to the elastic sheet (20).

10. The light treatment system (10, 12, 14) according to claim 9, wherein the joints (34) comprise a predefined level of friction for maintaining a shape of the deformable rim (30) after reshaping.

11. The light treatment system (10, 12, 14) according to claim 1, wherein the light treatment system (10, 12, 14) comprises a translucent cover sheet (80) arranged between the elastic sheet (20) and the treatment area (100).

12. The light treatment system (10, 12, 14) according to claim 11, wherein the translucent cover sheet (80) is removably attached to the light treatment system (10, 12, 14).

13. The light treatment system (10, 12, 14) according to claim 11, wherein the light treatment system (10, 12, 14) comprises fastening means (90) for attaching the system to a part of the human or animal body comprising the treatment area (100).

14. Light treatment system (10, 12, 14) as claimed in claim 1, wherein the light treatment system (10, 12, 14) comprises a control circuit (110) being configured for controlling the light emitting means (40, 42, 44) for emitting light towards the treatment area (100), the control circuit (110) being configured for controlling an intensity and/or a duration and/or a wavelength and/or a frequency in a pulse-mode operation and/or a dead-time in a pulse-mode operation.

15. Light treatment system (10, 12, 14) as claimed in claim 1, wherein the light treatment system (10, 12, 14) comprises vacuum means (120) for applying a reduced pressure inside a sealed environment (125) comprising the elastic sheet (20), the deformable rim (30) and the treatment area (100).
